(19) United States
(12) Patent Application Publication
(10) Pub. No.: US 2014/0290338 A1
(43) Pub. Date: Oct. 2, 2014

(54) HYDROGEN GAS SENSOR AND METHOD FOR MANUFACTURING THE SAME

(71) Applicant: Intellectual Discovery Co., Ltd., Seoul (KR)

(72) Inventors: Jung-Sik KIM, Seoul (KR); Beom-Jun KIM, Seoul (KR)

(73) Assignee: Intellectual Discovery Co., Ltd., Seoul (KR)

(21) Appl. No.: 13/904,116

(22) Filed: May 29, 2013

Related U.S. Application Data
(60) Provisional application No. 61/825,146, filed on May 20, 2013.

(30) Foreign Application Priority Data
Mar. 27, 2013 (KR) 10-2013-0033110

(51) Int. Cl. G01N 33/00; H01L 29/66

(52) U.S. Cl. CPC G01N 33/0009; H01L 29/66969
USPC 73/31.06; 438/49

(57) ABSTRACT

A hydrogen sensor and a method of manufacturing the same are provided. The hydrogen sensor includes i) a substrate, ii) a first metal oxide semiconductor that is formed in the substrate, and iii) a second metal oxide semiconductor that is separated from the first metal oxide semiconductor and that is formed in the substrate. The first metal oxide semiconductor includes i) a source electrode that is positioned on the substrate, ii) a drain electrode that is positioned on the substrate, iii) a channel layer that connects the source electrode and the drain electrode, iv) a gate insulating layer that is positioned on the channel layer, v) a gate electrode that is positioned on the gate insulating layer, and vi) a plurality of nano metal catalyst protrusions that are formed at an outside surface of the gate electrode to be applied to contact with hydrogen.

### CROSS-REFERENCE TO RELATED APPLICATION

This application is related to Korean Patent Application No. 2013-0033110 that was filed in the Korean Industrial Property Office at Mar. 27, 2013, “Gas Sensing Characteristics of Low-powered Dual MOSFET Hydrogen sensors”, which are a material that was announced at IMCS 2012 The 14th International meeting on Chemical Sensors that were opened at May 20, 2012, “Dual MOSFET Hydrogen Sensors with Thermal Island Structure” that was announced at IC-MAST 2012, 2nd International Conference on Materials & Applications for sensors & Transducers at May 24, 2012, A MEMS-type Micro Sensor for Hydrogen Gas Detection” that was announced at Nanotech Conferences & Expo 2012 at Jun. 18, 2012, and “Dual MOSFET Hydrogen sensors with Thermal Island Structure”, which is a treatise that was issued at pp. 93-96 of Key Engineering Materials Vol. 543 at Mar. 11, 2013.

### BACKGROUND OF THE INVENTION

(a) Field of the Invention

The present invention relates to a hydrogen sensor and a method of manufacturing the same. More particularly, the present invention relates to a hydrogen sensor having high sensitivity and excellent reliability and a method of manufacturing the same.

(b) Description of the Related Art

Due to environment contamination and resource exhaustion according to use of fossil fuel, energy that can replace the fossil fuel has been in the spotlight. For example, as energy that can replace fossil fuel, hydrogen has been in the spotlight and various research and development for commercially using hydrogen have been performed. However, when a predetermined concentration or more of hydrogen is exposed at the air, there is a problem that hydrogen easily explodes due to combustibility. Therefore, in order to easily use hydrogen energy, it is necessary to fast and accurately sense hydrogen leakage.

In order to sense hydrogen leakage, a hydrogen sensor is used. The hydrogen sensor senses hydrogen using a change of an electric signal according to a reaction with hydrogen of a metal or a semiconductor. Particularly, in order to accurately and fast sense hydrogen, a hydrogen sensor including a structure and a material having high reactivity to hydrogen is requested.

### SUMMARY OF THE INVENTION

A hydrogen sensor having advantages of precisely measuring a change of a hydrogen gas amount is provided. In addition, a method of manufacturing the hydrogen sensor is provided.

An exemplary embodiment of the present invention provides a hydrogen sensor including: i) a substrate; ii) a first metal oxide semiconductor that is formed in the substrate; and iii) a second metal oxide semiconductor that is separated from the first metal oxide semiconductor and that is formed in the substrate. The first metal oxide semiconductor includes i) a source electrode that is positioned on the substrate; ii) a drain electrode that is positioned on the substrate; iii) a channel layer that connects the source electrode and the drain electrode; iv) a gate insulating layer that is positioned on the channel layer; v) a gate electrode that is positioned on the gate insulating layer; and vi) a plurality of nano metal catalyst protrusions that are formed at an outside surface of the gate electrode to be applied to contact with hydrogen.

An average particle size of the plurality of nano metal catalyst protrusions may be 0 to 1000 nm. Preferably, an average particle size of the plurality of nano metal catalyst protrusions may be 50 nm to 500 nm. At least one nano metal catalyst protrusion of the plurality of nano metal catalyst protrusions may be a hollow space type. The plurality of nano metal catalyst protrusions may include at least one metal that is selected from a group consisting of palladium, iridium, ruthenium, and platinum or an alloy containing the metal. The hydrogen sensor may further include an insulating layer in which a sensing area including the first metal oxide semiconductor and the second metal oxide semiconductor is formed and that is provided on the substrate while surrounding the sensing area, wherein the insulating layer may be exposed to the outside toward a lower portion of the edge of the sensing area. The gate insulating layer and the insulating layer may be made of the same material. An average thickness of a non-sensing area surrounding the sensing area may be larger than that of the sensing area.

The hydrogen sensor may further include a passivation layer that is positioned under a substrate of the non-sensing area. A thickness of the substrate that is included in the sensing area may be 2 μm to 20 μm. A thickness of the substrate that is included in the non-sensing area may be 300 μm to 500 μm.

The hydrogen sensor may further include a passivation layer that is positioned on the source electrode, the drain electrode, the gate insulating layer, and the gate electrode, and the passivation layer may have an opening that exposes the plurality of nano metal catalyst protrusions to the outside. The passivation layer may cover the second metal oxide semiconductor to block a contact between the hydrogen and the second metal oxide semiconductor.

At least one electrode of electrodes that are selected from a group consisting of the source electrode and the drain electrode may include a material that is selected from a group consisting of platinum, palladium, iridium, and ruthenium. The hydrogen sensor may further include a microheater that is positioned on the substrate and that is separated from the first metal oxide semiconductor and the second metal oxide semiconductor. The source electrode, the drain electrode, the gate electrode, and the microheater may be made of the same material. The gate electrode and the plurality of nano metal catalyst protrusions may be integrally formed. Another passivation layer may be positioned under the substrate.

Another embodiment of the present invention provides a method of manufacturing a hydrogen sensor, the method including: i) providing a substrate; and ii) providing a separated first metal oxide semiconductor and second metal oxide semiconductor on the substrate. The providing of a separated first metal oxide semiconductor includes i) providing a separated source area and drain area by injecting ions into the substrate; ii) providing an oxide film on the substrate; iii) providing a source electrode and a drain electrode on the source area and the drain area, respectively, by masking the oxide film and providing a gate electrode on the oxide film; and iv) providing a plurality of nano metal catalyst protrusions on the gate electrode.

The providing of a plurality of nano metal catalyst protrusions may include i) providing resin beads on the gate electrode; ii) providing a metal catalyst on the resin beads; and iii) removing the resin beads by performing thermal treatment of the resin beads. At the providing of the resin beads, the resin beads may include at least one resin that is selected from a group consisting of polystyrene (PS), poly methylmethacrylate (PMMA), and poly dimethylsiloxane (PDMS). At the providing of a metal catalyst, the metal catalyst may be provided in a thin film form by sputtering or vacuum evaporation deposition on the resin beads. The method may further include partially removing a substrate that is included in a non-sensing area surrounding a sensing area including the first metal oxide semiconductor and the second metal oxide semiconductor.

A thickness of a hole that is formed by removing a substrate that is included in the non-sensing area may be 2 μm to 30 μm. The method may further include exposing the gate insulating layer to the outside by additionally removing the edge of the sensing area.

The method may further include charging a periphery of the source electrode, the drain electrode, and the gate electrode with a passivation layer. The providing of a gate electrode on the oxide film may include together providing a microheater on the oxide film. The providing of a plurality of nano metal catalyst protrusions may include i) providing an aluminum thin film; ii) providing a template including separated micro holes by anodizing the aluminum thin film; iii) charging a metal catalyst at the micro holes; and iv) providing a nano metal catalyst protrusion by removing the template.

A hydrogen concentration using a hydrogen sensor can be precisely measured. Further, hydrogen sensitivity can be largely improved using nano metal catalyst protrusions.

### BRIEF DESCRIPTION OF THE DRAWINGS

FIG. 1 is a schematic cross-sectional view of a hydrogen sensor according to a first exemplary embodiment of the present invention.

FIG. 2 is a flowchart schematically illustrating a method of manufacturing the hydrogen sensor of FIG. 1.

FIGS. 3 to 12 are cross-sectional views schematically illustrating each step of the method of manufacturing a hydrogen sensor of FIG. 2.

FIG. 13 is a schematic cross-sectional view of a hydrogen sensor according to a second exemplary embodiment of the present invention.

FIG. 14 is a flowchart schematically illustrating a method of manufacturing another nano metal catalyst protrusions of FIG. 13.

### DETAILED DESCRIPTION OF THE EMBODIMENTS

When it is said that any part is positioned “on” another part, it means the part is directly on the other part or above the other part with at least one intermediate part. In contrast, if any part is said to be positioned “directly on” another part, it means that there is no intermediate part between the two parts.

Technical terms used here are to only describe a specific exemplary embodiment and are not intended to limit the present invention. Singular forms used here include a plurality of forms unless phrases explicitly represent an opposite meaning. A meaning of “comprising” used in a specification embodies a specific characteristic, area, integer, step, operation, element and/or component and does not exclude presence or addition of another characteristic, area, integer, step, operation, element, component and/or group.

Terms representing relative space of “low” and “upper” may be used for more easily describing a relationship to another portion of a portion shown in the drawings. Such terms are intended to include other meanings or operations of a using apparatus together with a meaning that is intended in the drawings. For example, when an apparatus is inverted in the drawings, any portion described as disposed at a “low” portion of other portions is described as being disposed at an “upper” portion of other portions. Therefore, an illustrative term of “low” includes entire upper and lower directions. An apparatus may rotate by 90° or another angle, and a term representing relative space is accordingly analyzed.

Although not differently defined, entire terms including a technical term and a scientific term used here have the same meaning as a meaning that may be generally understood by a person of common skill in the art. It is additionally analyzed that terms defined in a generally used dictionary have a meaning corresponding to a related technology document and presently disclosed contents and are not analyzed as an ideal or very official meaning unless stated otherwise.

The present invention will be described more fully hereinafter with reference to the accompanying drawings, in which exemplary embodiments of the invention are shown. As those skilled in the art would realize, the described embodiments may be modified in various different ways, all without departing from the spirit or scope of the present invention.

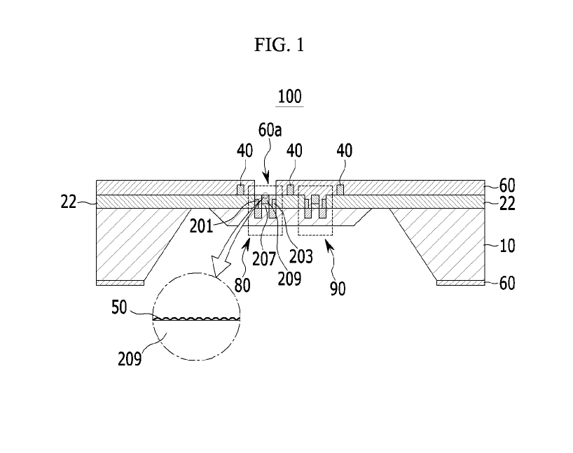

FIG. 1 is a cross-sectional view of a hydrogen sensor 100 according to a first exemplary embodiment of the present invention. An enlarged circle of FIG. 1 illustrates an enlarged gate electrode 209. A structure of the hydrogen sensor 100 of FIG. 1 illustrates the present invention and the present invention is not limited thereto. Therefore, a structure of the hydrogen sensor 100 may be changed to other forms.

As shown in FIG. 1, the hydrogen sensor 100 includes a substrate 10, a first metal oxide semiconductor 80, a second metal oxide semiconductor 90, and a microheater 40. In addition, the hydrogen sensor 100 may further include other elements, as needed. Meanwhile, an insulating layer 22 is positioned on the substrate 10, and a passivation layer 60 that covers the first metal oxide semiconductor 80 and the second metal oxide semiconductor 90 is positioned on the insulating layer 22. Because an opening 60a is formed in the passivation layer 60, a plurality of nano metal catalyst protrusions 50 are exposed to the outside through the opening 60a to sense hydrogen. By covering the second metal oxide semiconductor 90, the passivation layer 60 blocks a contact between hydrogen and the second metal oxide semiconductor 90. Further, in a lower portion of the substrate 10, another passivation layer 60 is positioned to passivate the substrate 10 from the outside for electrical ground. Therefore, an upper electrode (not shown), the first metal oxide semiconductor 80, the second metal oxide semiconductor 90, and the substrate 10 are connected and thus electrical damage does not occur.

The first metal oxide semiconductor 80 and the second metal oxide semiconductor 90 are formed in the substrate 10. That is, the first metal oxide semiconductor 80 and the second metal oxide semiconductor 90 may be each formed in the substrate 10 using a semiconductor process. The first metal oxide semiconductor 80 and the second metal oxide semiconductor 90 are separated from each other. The first metal oxide semiconductor 80 communicates with the outside through the opening 60a, but the second metal oxide semiconductor 90 is blocked from the outside by the passivation layer 60. Therefore, the first metal oxide semiconductor 80 functions as a sensing electrode, and the second metal oxide semiconductor 90 functions as a reference electrode. The first metal oxide semiconductor 80 and the second metal oxide semiconductor 90 are produced through the same structure and the same method, except for the nano metal catalyst protrusion 50. Therefore, by comparing the first metal oxide semiconductor 80 and the second metal oxide semiconductor 90, a hydrogen concentration is measured. As a result, by measuring and comparing a change amount of a current or a voltage that is together applied to the first metal oxide semiconductor 80 and the second metal oxide semiconductor 90, a hydrogen concentration is measured. Meanwhile, the microheater 40 is positioned on the substrate 10 to be positioned separately from the first metal oxide semiconductor 80 and the second metal oxide semiconductor 90. By appropriately heating the hydrogen sensor 100, the microheater 40 improves sensitivity of the first metal oxide semiconductor 80 to hydrogen.

Hereinafter, a structure of the first metal oxide semiconductor 80 of FIG. 1 will be described in detail. A structure of the second metal oxide semiconductor 90 of FIG. 1 is similar to that of the first metal oxide semiconductor 80 and therefore a detailed description thereof will be omitted.

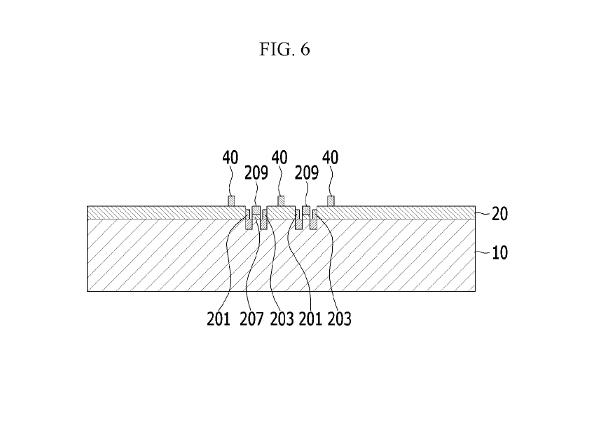

As shown in FIG. 1, the first metal oxide semiconductor 80 includes a source electrode 201, a drain electrode 203, a gate insulating layer 207, a gate electrode 209, and a plurality of nano metal catalyst protrusions 50. In addition, the first metal oxide semiconductor 80 may further include other constituent elements, as needed. The source electrode 201 and the drain electrode 203 are positioned on the substrate 10. A source area S (shown in FIG. 6, hereinafter the same) and a drain area D (shown in FIG. 6, hereinafter the same) are positioned in a lower portion of the source electrode 201 and the drain electrode 203, respectively. A channel area C (shown in FIG. 6, hereinafter the same) that connects the source area S and the drain area D is positioned between the source area S and the drain area D. Therefore, a current that is injected through the source electrode 201 flows to the drain area D through the channel area C and is output to the outside through the drain electrode 203.

The gate insulating layer 207 is positioned on the channel layer C, and the gate electrode 209 is positioned on the gate insulating layer 207. A current flowing through the channel area C is adjusted through a voltage that is applied to the gate electrode 209. The gate insulating layer 207 may be formed together with the same material as that of the insulating layer 22.

As shown in an enlarged circle of FIG. 1, a plurality of nano metal catalyst protrusions 50 are formed in an outside surface of the gate electrode 209 to contact with hydrogen. Therefore, by appropriately adjusting a temperature of the hydrogen sensor 100 through the microheater 40, the plurality of nano metal catalyst protrusions 50 react well with hydrogen. As a result, a hydrogen concentration can be precisely measured using the hydrogen sensor 100. Here, the hydrogen sensor 100 can measure entire hydrogen concentration of a gas form or an aqueous solution form.

The plurality of nano metal catalyst protrusions 50 are formed in a micro structure of a nano scale instead of being formed in a bulk form. That is, an average particle size of the plurality of nano metal catalyst protrusions 50 may be 0 to 1000 nm. If the average particle size of the plurality of nano metal catalyst protrusions 50 is too large, a surface area thereof slightly increases, and thus a hydrogen sensing effect is not large. Therefore, it is necessary to adjust the average particle size of the plurality of nano metal catalyst protrusions 50 to the foregoing range. More preferably, the average particle size of the plurality of nano metal catalyst protrusions 50 may be adjusted to 50 nm to 500 nm. By adjusting the average particle size of the plurality of nano metal catalyst protrusions 50 to foregoing range, a hydrogen detect effect of the hydrogen sensor 100 can be optimized. Because a surface area of the gate electrode 209 largely increases due to the plurality of nano metal catalyst protrusions 50, hydrogen of a low concentration can be precisely sensed. For this, a specific surface area of the plurality of nano metal catalyst protrusions 50 may be about 1.5 times to 5 times greater than that of a general flat film. If the specific surface area of the plurality of nano metal catalyst protrusions 50 is too small, hydrogen sensitivity is deteriorated. In addition, if the specific surface area of the plurality of nano metal catalyst protrusions 50 is too large, structural stability of the plurality of nano metal catalyst protrusions 50 is deteriorated. Therefore, it is preferable to adjust a specific surface area of the plurality of nano metal catalyst protrusions 50 to the foregoing range.

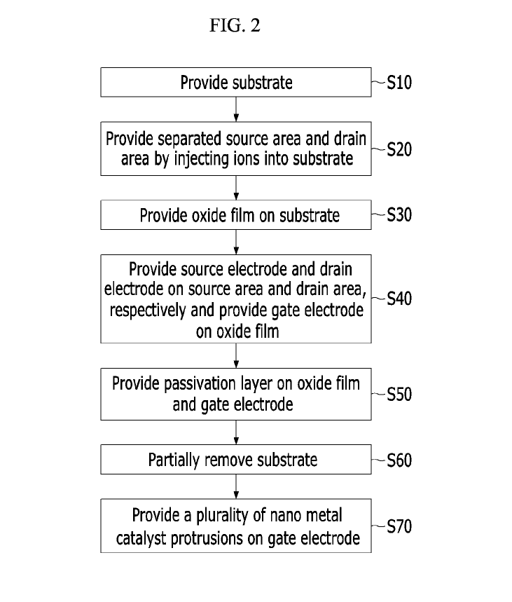

As shown in an enlarged circle of FIG. 1, the plurality of nano metal catalyst protrusions 50 having the empty inside, i.e., having a hollow space structure may be formed. As a result, a production cost of the plurality of nano metal catalyst protrusions 50 is less consumed and hydrogen sensitivity can be further improved. Meanwhile, the plurality of nano metal catalyst protrusions 50 may be integrally formed with the gate electrode 209. Therefore, when manufacturing the gate electrode 209, by together manufacturing the plurality of nano metal catalyst protrusions 50, a process can be simplified. Hereinafter, a process of manufacturing the hydrogen sensor 100 of FIG. 1 will be described in detail with reference to FIG. 2.

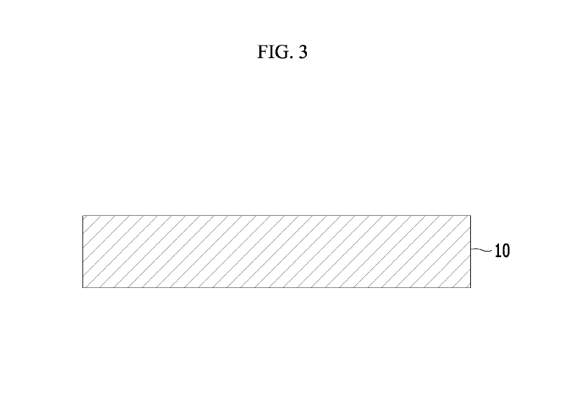

FIG. 2 schematically shows a method of manufacturing the hydrogen sensor 100 of FIG. 1. The method of manufacturing the hydrogen sensor of FIG. 2 illustrates the present invention and the present invention is not limited thereto. Therefore, a method of manufacturing a hydrogen sensor may be changed to other forms. FIGS. 3 to 12 illustrate each step of FIG. 2 and hereinafter, FIG. 2 will be described in detail with reference to FIGS. 3 to 12.

The method of manufacturing the hydrogen sensor of FIG. 2 includes step of providing a substrate (S10), step of providing a separated source area and drain area by injecting ions into the substrate (S20), and step of providing an oxide film on the substrate (S30), step of providing a source electrode and a drain electrode on the source area and the drain area, respectively and providing a gate electrode on the oxide film (S40), step of providing a passivation layer on the oxide film and the gate electrode (S50), step of partially removing the substrate (S60), and step of providing a plurality of nano metal catalyst protrusions on the gate electrode (S70). In addition, a method of manufacturing a hydrogen sensor may further include other steps or may omit some steps of the foregoing steps. The foregoing method of manufacturing a hydrogen sensor is a method of manufacturing the first metal oxide semiconductor, but the second metal oxide semiconductor may be produced with the same method, except for step S70. That is, because the second metal oxide semiconductor is separated from and is positioned parallel to the first metal oxide semiconductor, the second metal oxide semiconductor may be formed simultaneously with the first metal oxide semiconductor.

First, the substrate 10 is provided at step S10 of FIG. 2 (shown in FIG. 3). As a material of the substrate 10, p-type silicon, n-type silicon, or silicon oxide may be used. In a following process, ions are injected into the substrate 10 and thus a source area, a channel area, and a drain area are formed.

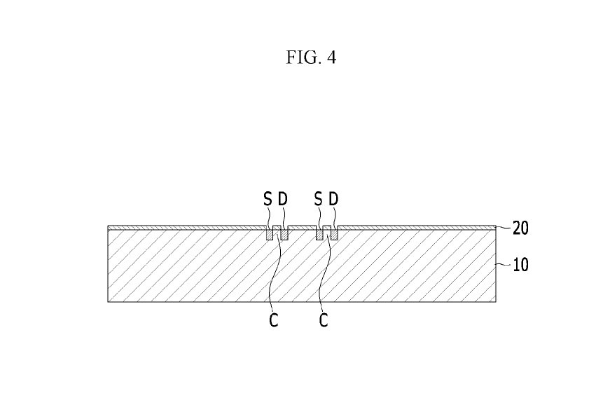

At step S20 of FIG. 2, after an oxide film 20 is patterned, by injecting ions, a separated source area S and drain area D are provided (shown in FIG. 4). A channel area C is formed between the source area S and the drain area D. After covering the oxide film 20 with a mask in which a pattern is formed, by exposing and developing the oxide film 20, a pattern for forming the source area S and the drain area D may be formed on the oxide film 20. By selectively injecting ions into only the substrate 10 that is positioned at a pattern forming portion, the source area S and the drain area D are formed.

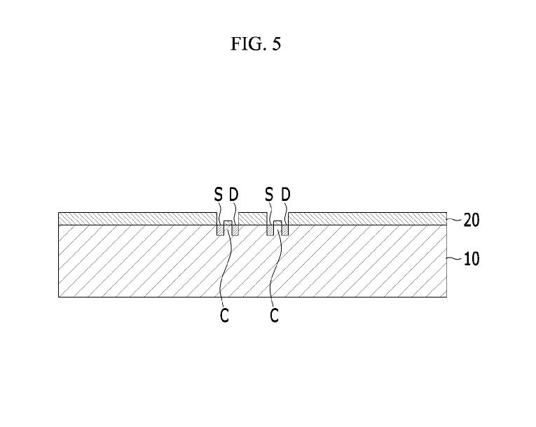

Next, at step S30 of FIG. 2, the oxide film 20 is more thickly provided on the substrate 10 (shown in FIG. 5). That is, the insulating oxide film 20 may be formed on the substrate 10 through a method of heating a substrate. After step S30 is complete, a mask may be removed.

At step S40 of FIG. 2, the source electrode 201 and the drain electrode 203 are provided on the source area S and the drain area D, respectively, and the gate electrode 209 is provided on the oxide film 20 (shown in FIG. 6). Here, the source electrode 201, the drain electrode 203, and the gate electrode 209 are simultaneously formed. The microheater 40 may be formed together with the source electrode 201, the drain electrode 203, and the gate electrode 209. That is, after the insulating layer film 20 is covered with another mask in which a pattern is formed, by depositing a metal, the source electrode 201, the drain electrode 203, the gate electrode 209, and the microheater 40 are simultaneously formed. In this case, as the source electrode 201 and the drain electrode 203 are connected to the gate electrode 209, the mask separates the gate electrode 209, the source electrode 201, and the drain electrode 203 so that a short phenomenon does not occur.

The source electrode 201, the drain electrode 203, the gate electrode 209, or the microheater 40 may be formed using a material of a metal of platinum, palladium, iridium, or ruthenium or an alloy containing such a metal. Because the source electrode 201, the drain electrode 203, the gate electrode 209, or the microheater 40 is made of the foregoing material, the source electrode 201, the drain electrode 203, the gate electrode 209, or the microheater 40 has excellent efficiency and particularly, the gate electrode 209 has excellent sensitivity to hydrogen. Therefore, hydrogen sensing efficiency of the hydrogen sensor 100 (FIG. 1) can be largely improved.

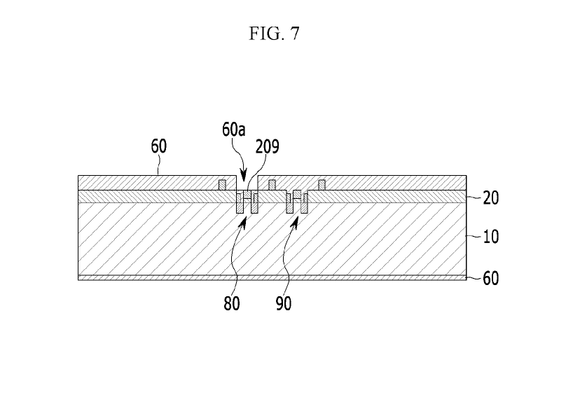

Next, at step S50 of FIG. 2, a passivation layer 60 is provided on the oxide film 20 and the gate electrode 209 (shown in FIG. 7). That is, the passivation layer 60 is formed so that the remaining portions of the hydrogen sensor 100 (FIG. 1), except for the first metal oxide semiconductor 80 does not contact with hydrogen, and the opening 60a is formed only on the first metal oxide semiconductor 80 through patterning. For example, after exposing only an area in which the source electrode 201, the drain electrode 203, and the gate electrode 209 exist to the outside using a mask, the passivation layer 60 may be formed. The passivation layer 60 fills a space at which the source electrode 201, the drain electrode 203, and the gate electrode 209 are positioned. When the mask that is positioned on the gate electrode 209 is removed, the opening 60a is formed on the gate electrode 209 and thus the gate electrode 209 is exposed to the outside to contact with hydrogen.

Meanwhile, even in a lower portion of the substrate 10, the passivation layer 60 is formed. The passivation layer 60 may be used as a mask layer that prevents etching of a portion other than an area to etch the substrate 10 in a process of step S60 and step S70 of FIG. 2. The passivation layer 60 is formed using a method of low pressure chemical vapor deposition (LPCVD) that can be simultaneously deposited in an upper portion and a lower portion. At step S50, as only the first metal oxide semiconductor 80 reacts with hydrogen, a change occurs in a voltage and a current and thus a hydrogen density is measured through a comparison with the second metal oxide semiconductor 90.

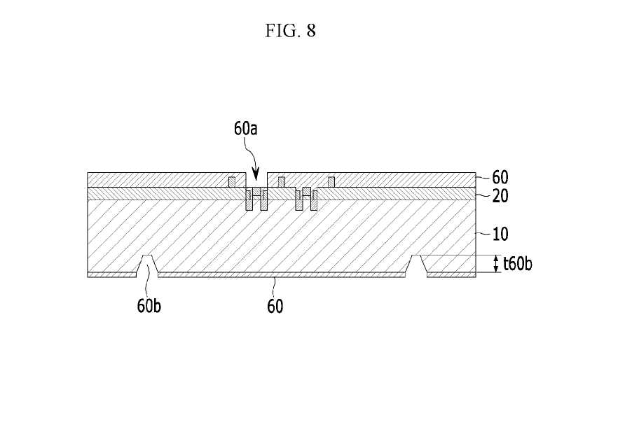

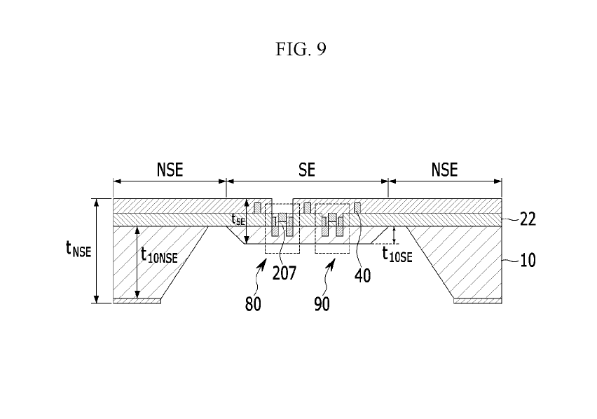

At step S60 of FIG. 2, the substrate 10 is partially removed (shown in FIG. 8). That is, in order to enhance hydrogen sensitivity of the hydrogen sensor 100, it is necessary to partially remove the substrate in order to form a portion at which the first metal oxide semiconductor 80 and the second metal oxide semiconductor 90 are positioned like an island. The hydrogen sensor 100 (shown in FIG. 1) includes a sensing area (SE) (shown in FIG. 9) and a non-sensing area (NSE) (shown in FIG. 9) surrounding the same. The sensing area SE includes the first metal oxide semiconductor 80 and the second metal oxide semiconductor 90. The non-sensing area NSE surrounds the sensing area SE.

Therefore, as shown in FIG. 8, the substrate 10 is firstly partially removed through chemical etching or a micro mechanical processing. That is, a substrate that is included in the NSE surrounding the sensing area SE including the first metal oxide semiconductor 80 and the second metal oxide semiconductor 90 is partially removed. As a result, while the passivation layer 60 and the substrate 10 are partially etched, a hole 60b is firstly formed. Here, a thickness t60b of the hole 60b may be 2 μm to 30 μm. If the thickness t60b of the hole 60b is too large, the substrate 10 is so much removed and thus when secondarily removing the substrate 10, the substrate of the sensing area SE is also etched and thus a source area and a drain area of an oxide semiconductor may not be formed. In addition, if the thickness t60b of the hole 60b is too small, when the substrate 10 is secondarily removed, the hydrogen sensor 100 (shown in FIG. 1) may not be formed in an island structure. Therefore, the thickness t60b of the hole 60b is adjusted to the foregoing range.

FIG. 9 illustrates a state in which the substrate 10 is secondarily removed. Here, the oxide film 20 of FIG. 8 is divided into the insulating layer 22 and the gate insulating layer 207 of FIG. 9. The insulating layer 22 is provided on the substrate 10 while surrounding the sensing area SE. The insulating layer 22 is exposed to the outside toward a lower portion of the edge of the non-sensing area SE while the substrate 10 is removed. That is, when the substrate 10 that is included in the sensing area SE is partially removed, the hydrogen sensor 100 is formed in an island form and thus by partially heating only the sensing area SE by the microheater 40, hydrogen sensitivity can be enhanced. That is, an average thickness t_{NSE} of the non-sensing area NSE that surrounds the sensing area SE is larger than an average thickness t_{SE} of the sensing area SE.

In more detail, a thickness t_{10SE} of the substrate 10 that is included in the sensing area SE may be 2 μm to 30 μm. If the thickness t_{10SE} of the substrate 10 is too large, power consumption of a sensor may increase and a load of a membrane increases and thus the membrane may be structurally weak. If the thickness t_{iosE} of the substrate 10 is too small, it is difficult that a source area and a drain area of an oxide semiconductor are positioned at the inside of the sensing area SE, and it is difficult to secure an etching processing process condition. Therefore, it is preferable to maintain the thickness t_{10SE} of the substrate 10 to the foregoing range. The thickness t_{10NSE} of the substrate 10 that is included in the non-sensing area NSE may be 300 μm to 500 μm. If the thickness t_{10NSE} of the substrate 10 is too large, a size of the hydrogen sensor 100 (shown in FIG. 1) is too large and thus consumption power increases and thus it is unpreferable in view of manufacturing. If the thickness t_{10NSE} of the substrate 10 is too small, a chip frame for supporting an entire structure of a sensor is too thin and thus it is difficult to secure stability of the sensor. Therefore, it is preferable to adjust the thickness t_{10NSE} of the substrate 10 to the foregoing range.

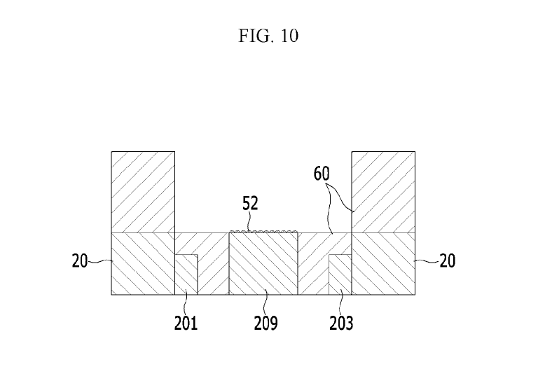

When returning again to FIG. 2, the plurality of nano metal catalyst protrusions 50 are provided on the gate electrode 209 (S70). In FIGS. 10 to 12, step of providing the plurality of nano metal catalyst protrusions 50 are sequentially illustrated, and for convenience of description, FIGS. 10 to 12 enlarge and illustrate the gate electrode 209 of FIG. 9 and a periphery thereof.

Step of providing the plurality of nano metal catalyst protrusions 50 includes i) step of providing resin beads 52 on the gate electrode 209, ii) step of providing a metal catalyst 54 on the resin beads 52, and iii) step of removing the resin beads 52 by performing heat treatment of a hydrogen sensor. In addition, step of providing the plurality of nano metal catalyst protrusions 50 may further include other steps, as needed.

As shown in FIG. 10, the resin beads 52 are provided on the gate electrode 209. Here, the resin beads 52 are produced using a resin such as polystyrene (PS), poly-methylmethacrylate (PMMA) or poly-dimethylsiloxane (PDMS). Because such a kind of resin beads 52 are volatilized at 400° C. or more, the resin beads 52 are appropriate to produce the plurality of nano metal catalyst protrusions 50. The resin beads 52 may be produced with a method of spin coating or deep coating by distributing resin beads within a solution such as a water-soluble solvent in a suspension form. In order to provide the resin beads 52 on the gate electrode 209, the remaining portions, except for a portion in which the resin beads 52 are formed may be entirely blocked through masking.

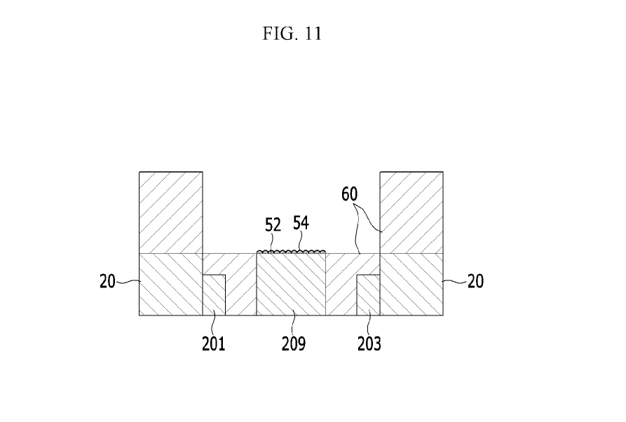

Next, as shown in FIG. 11, the metal catalyst 54 is provided on the resin beads 52. The metal catalyst 54 may be provided in a thin film form on the resin beads 52 by sputtering or vacuum evaporation deposition. Further, the metal catalyst 54 may be formed through a method of room temperature deposition. As a material of the metal catalyst 54, at least one metal selected from a group consisting of palladium, iridium, ruthenium, and platinum having excellent reactivity with hydrogen or an alloy containing the foregoing metal may be used.

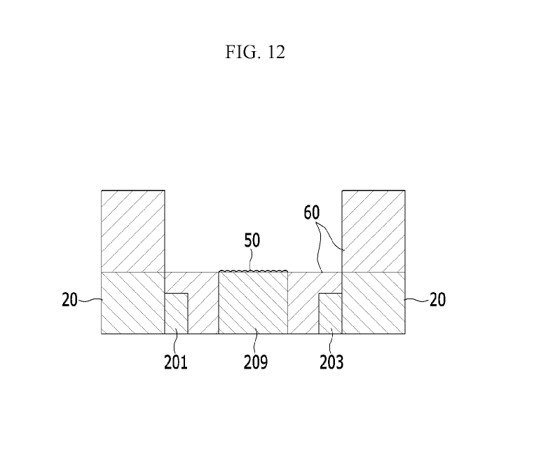

As shown in FIG. 12, by performing heat treatment of the resin beads 52, the resin beads 52 are removed. In this case, the resin beads 52 are evaporated by heat treatment in a temperature of 400° C. or more. As a result, the plurality of nano metal catalyst protrusions 50 that may be formed in a hollow space type on the gate electrode 209 may be produced.

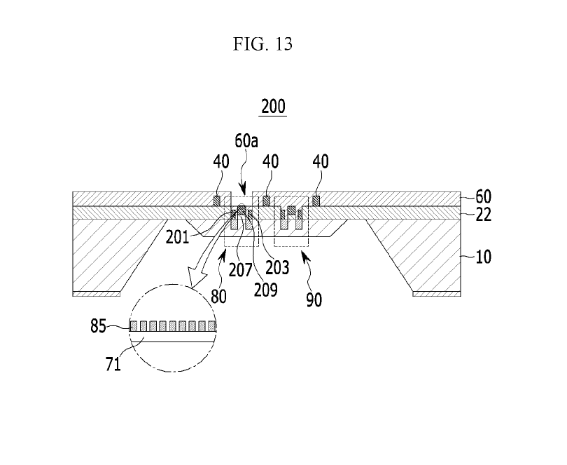

FIG. 13 partially enlarges and illustrates a hydrogen sensor 200 that is produced according to a second exemplary embodiment of the present invention. In an enlarged circle of FIG. 13, another nano metal catalyst protrusions 84 that are formed on a gate electrode 209 are enlarged and illustrated. Since a structure of the hydrogen sensor 200 of FIG. 13 is similar to that of the hydrogen sensor 100 of FIG. 1, like numerals refer to like elements and a detailed description thereof will be omitted.

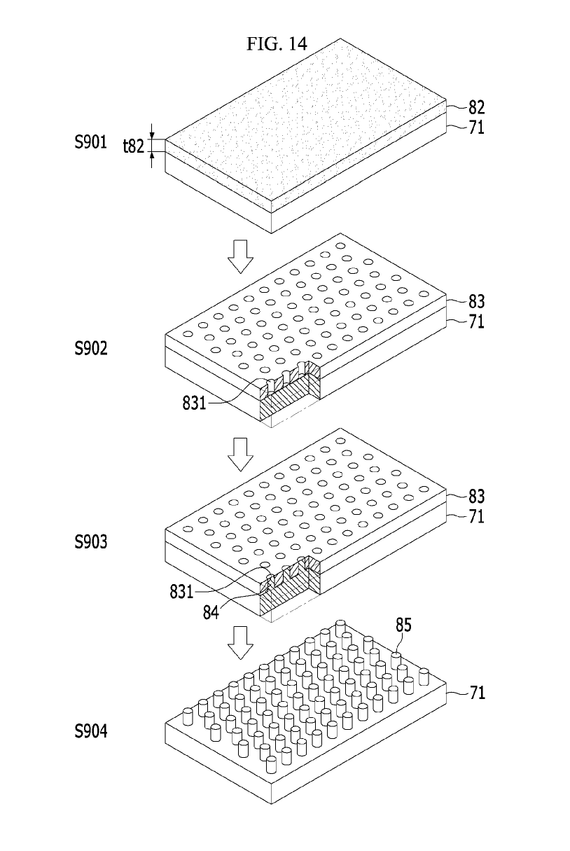

As shown in FIG. 13, another nano metal catalyst protrusions 85 are formed on the gate electrode 209 of the hydrogen sensor 200. Here, the nano metal catalyst protrusions 85 are formed on a template 71 that is produced through an anodizing process. Hereinafter, an anodizing process of producing the template 71 will be described in detail with reference to FIG. 14.

FIG. 14 schematically illustrates a flowchart of a method of manufacturing another nano metal catalyst protrusions 85 of FIG. 13. For convenience of description, step S902 and step S903 of FIG. 14 are illustrated through a partial cross-sectional view. A method of manufacturing another nano metal catalyst protrusions 85 of FIG. 14 illustrates the present invention and the present invention is not limited thereto. Therefore, a method of manufacturing another nano metal catalyst protrusions 85 may be changed to other forms.

As shown in FIG. 14, a method of manufacturing another nano metal catalyst protrusions 85 includes step of providing an aluminum thin film 82 (S901), step of providing a template 83 including separated micro holes 831 by anodizing the aluminum thin film 82 (S902), step of charging a metal catalyst 84 at the micro holes 831, and step of providing nano metal catalyst protrusions 85 by removing the template 83. In addition, a method of manufacturing another nano metal catalyst protrusions 85 may further include other steps, as needed.

First, the aluminum thin film 82 to use as a template is provided on a base plate 71 (S901). A thickness t82 of the aluminum thin film 82 may be 2 μm to 5 μm. If the thickness t82 of the aluminum thin film 82 is too large, a thin film deposition time and a process cost increase and a pore occurs at the inside or a surface of the thin film and thus the following anodizing process may be difficult. Further, after anodizing, a deposited metal catalyst is deposited only at an anodizing surface and thus a nano metal catalyst protrusion is not well formed. If a thickness t82 of the aluminum thin film 82 is too small, after anodizing, a deposited metal catalyst is continuously formed and thus nano metal catalyst protrusions are not formed. Therefore, it is preferable to adjust the thickness t82 of the aluminum thin film 82 to the foregoing range, for example, the thickness t82 of the aluminum thin film 82 may be 2 μm. Here, aluminum purity of the aluminum thin film 82 may be 99.9999%. The aluminum thin film 82 is anodized (S902). That is, the aluminum thin film 82 is used as a positive electrode in an aqueous solution such as C₂H₂O₄, and platinum is used as a negative electrode and then a voltage is applied. As a result, the aluminum thin film 82 is anodized and is converted to the template 83. In this case, the micro holes 831 are formed in the template 83 and thus it is preferable to perform an anodizing process in a level to expose a surface of the base plate 71.

The metal catalyst 84 is charged at the micro holes 831 (S903). Therefore, the metal catalyst 84 is charged at the template 83. The metal catalyst 84 may be produced with a method of sputtering or deposition, but a method of producing the metal catalyst 84 is not limited thereto, and it is preferable that a deposition thickness is about 10% to 20% of a template thickness.

Finally, by removing the template 83, the nano metal catalyst protrusions 85 are formed (S904). The template 83 may be removed by dipping the template 83 in an acid solution such as chrome acid and phosphoric acid or by selectively etching using a gas such as chlorine (Cl₂) or boron chloride (BClx). By attaching the produced base plate 71 and the nano metal catalyst protrusions 85 on the gate electrode 209 (shown in FIG. 13), the hydrogen gas sensor 200 (shown in FIG. 13) is produced.

While this invention has been described in connection with what is presently considered to be practical exemplary embodiments, it is to be understood that the invention is not limited to the disclosed embodiments, but, on the contrary, is intended to cover various modifications and equivalent arrangements included within the spirit and scope of the appended claims.

What is claimed is:

1. A hydrogen sensor, comprising:
a substrate;
a first metal oxide semiconductor that is formed in the substrate; and
a second metal oxide semiconductor that is separated from the first metal oxide semiconductor and that is formed in the substrate,
wherein the first metal oxide semiconductor comprises
a source electrode that is positioned on the substrate;
a drain electrode that is positioned on the substrate;
a channel layer that connects the source electrode and the drain electrode;
a gate insulating layer that is positioned on the channel layer;
a gate electrode that is positioned on the gate insulating layer; and
a plurality of nano metal catalyst protrusions that are formed at an outside surface of the gate electrode to be applied to contact with hydrogen.

2. The hydrogen sensor of claim 1, wherein an average particle size of the plurality of nano metal catalyst protrusions is 0 to 1000 nm.

3. The hydrogen sensor of claim 2, wherein an average particle size of the plurality of nano metal catalyst protrusions is 50 nm to 500 nm.

4. The hydrogen sensor of claim 1, wherein at least one nano metal catalyst protrusion of the plurality of nano metal catalyst protrusions is a hollow space type.

5. The hydrogen sensor of claim 1, wherein the plurality of nano metal catalyst protrusions comprise at least one metal that is selected from a group consisting of palladium, iridium, ruthenium, and platinum or an alloy containing the metal.

6. The hydrogen sensor of claim 1, further comprising an insulating layer in which a sensing area comprising the first metal oxide semiconductor and the second metal oxide semiconductor is formed and that is provided on the substrate while surrounding the sensing area,
wherein the insulating layer is exposed to the outside toward a lower portion of the edge of the sensing area.

7. The hydrogen sensor of claim 6, wherein the gate insulating layer and the insulating layer are made of the same material.

8. The hydrogen sensor of claim 7, wherein an average thickness of a non-sensing area surrounding the sensing area is larger than that of the sensing area.

9. The hydrogen sensor of claim 8, wherein the hydrogen sensor further comprises a passivation layer that is positioned under a substrate of the non-sensing area.

10. The hydrogen sensor of claim 9, wherein a thickness of the substrate that is included in the sensing area is 2 μm to 20 μm.

11. The hydrogen sensor of claim 10, wherein a thickness of the substrate that is included in the non-sensing area is 300 μm to 500 μm.

12. The hydrogen sensor of claim 1, wherein the hydrogen sensor further comprises a passivation layer that is positioned on the source electrode, the drain electrode, the gate insulating layer, and the gate electrode, and the passivation layer has an opening that exposes the plurality of nano metal catalyst protrusions to the outside.

13. The hydrogen sensor of claim 12, wherein the passivation layer covers the second metal oxide semiconductor to block a contact between the hydrogen and the second metal oxide semiconductor.

14. The hydrogen sensor of claim 1, wherein at least one electrode of electrodes that are selected from a group consisting of the source electrode and the drain electrode comprises a material that is selected from a group consisting of platinum, palladium, iridium, and ruthenium.

15. The hydrogen sensor of claim 1, wherein the hydrogen sensor further comprises a microheater that is positioned on the substrate and that is separated from the first metal oxide semiconductor and the second metal oxide semiconductor.

16. The hydrogen sensor of claim 15, wherein the source electrode, the drain electrode, the gate electrode, and the microheater are made of the same material.

17. The hydrogen sensor of claim 1, wherein the gate electrode and the plurality of nano metal catalyst protrusions are integrally formed.

18. The hydrogen sensor of claim 1, wherein another passivation layer is positioned under the substrate.

19. A method of manufacturing a hydrogen sensor, the method comprising:
providing a substrate; and
providing a separated first metal oxide semiconductor and second metal oxide semiconductor on the substrate,
wherein the providing of a separated first metal oxide semiconductor comprises
providing a separated source area and drain area by injecting ions into the substrate;
providing an oxide film on the substrate;
providing a source electrode and a drain electrode on the source area and the drain area, respectively, by masking the oxide film and providing a gate electrode on the oxide film; and
providing a plurality of nano metal catalyst protrusions on the gate electrode.

20. The method of claim 19, wherein the providing of a plurality of nano metal catalyst protrusions comprises
providing resin beads on the gate electrode;
providing a metal catalyst on the resin beads; and
removing the resin beads by performing thermal treatment of the resin beads.

21. The method of claim 19, wherein at the providing of resin beads, the resin beads comprises at least one resin that is selected from a group consisting of polystyrene (PS), poly methylmethacrylate (PMMA), and poly dimethylsiloxane (PDMS).

22. The method of claim 19, wherein at the providing of a metal catalyst, the metal catalyst is provided in a thin film form by sputtering or vacuum evaporation deposition on the resin beads.

23. The method of claim 19, further comprising partially removing a substrate that is included in a non-sensing area surrounding a sensing area comprising the first metal oxide semiconductor and the second metal oxide semiconductor.

24. The method of claim 23, wherein a thickness of a hole that is formed by removing a substrate that is included in the non-sensing area is 2 μm to 30 μm.

25. The method of claim 23, further comprising exposing the gate insulating layer to the outside by additionally removing the edge of the sensing area.

26. The method of claim 19, further comprising charging a periphery of the source electrode, the drain electrode, and the gate electrode with a passivation layer.

27. The method of claim 19, wherein the providing of a gate electrode on the oxide film comprises together providing a microheater on the oxide film.

28. The method of claim 19, wherein the providing of a plurality of nano metal catalyst protrusions comprises
providing an aluminum thin film;
providing a template comprising separated micro holes by anodizing the aluminum thin film;
charging a metal catalyst at the micro holes; and
providing a nano metal catalyst protrusion by removing the template.
